(19) United States
(12) Patent Application Publication
(10) Pub. No.: US 2011/0112644 A1
(43) Pub. Date: May 12, 2011

(54) DISC PROSTHETIC IMPLANT DEVICE

(76) Inventors: Boris ZILBERSTEIN, Haifa (IL); Netanel Benichou, Hof-Carmel (IL); Oded Elish, Kiryat Tivon (IL)

(21) Appl. No.: 12/617,094

(22) Filed: Nov. 12, 2009

(51) Int. Cl. A61F 2/44

(52) U.S. Cl. 623/17.15

(57) ABSTRACT

An intervertebral prosthetic implant device is described. The device includes a first plate with an axis support for supporting a rolling element substantially separate from the remainder of the first plate, designed to allow rolling of the rolling element. The device also includes a second plate that includes a groove designed to accommodate the rolling element, facilitating rotating and rolling motion of the second plate with respect to the first plate by rolling over the rolling element.

### FIELD OF THE INVENTION

The present invention relates to prosthetic implant devices. More particularly, the present invention relates to a disc prosthetic device.

### BACKGROUND OF THE INVENTION

Spinal disc replacement may be offered as a possible treatment for patients suffering from degeneration, herniation, or other impairment an intervertebral disc. The intervertebral disc is located between adjacent vertebral bodies in the ventral portion of the spine. A healthy intervertebral disc serves to separate and cushions the adjacent vertebral bodies. When the function of the intervertebral disc is impaired, the space between the vertebrae separated by the impaired disc may be reduced, affecting the movement of the vertebrae. The change in movement of the vertebrae may cause pain or other problems for the patient. One method used for treating such conditions is spinal fusion. In spinal fusion, two or more vertebrae are fused such that relative motion between the fused vertebrae is prevented. Thus, the unnatural motion of the vertebrae is prevented.

Disc replacement may serve as an alternative treatment to spinal fusion. In disc replacement, one or more disc replacement devices are implanted between the vertebral bodies in place of the impaired disc. Disc replacement devices may provide some of the function of a healthy disc. The provided function may include preserving a normal distance between the vertebrae and preventing unnatural motion of the vertebrae. A potential advantage of disc replacement over spinal fusion is that with disc replacement, depending on the design of the disc replacement device, the vertebrae may retain some of the freedom of movement of a healthy spine.

Since a disc replacement device is to be implanted between vertebral bodies in the ventral portion of the spine, some such devices are designed to be inserted directly between the vertebral bodies. Insertion via anterior access, avoids traversing the dorsal part of the spine that contains the spinal cord. Anterior access enables insertion of a replacement device whose dimensions are similar to those of the disc being replaced. However, anterior access, requiring traversal of the entire body cavity anterior to the spine, may be considered a more invasive option than posterior access. In posterior access, the replacement device is inserted via the dorsal portion of the spine. With posterior access, a replacement device inserted via a path that traverses the dorsal portion of the spine. The insertion path is lateral to, and avoids contact with, the spinal cord. Implantation of the device may require performing a partial laminectomy to remove part of the lamina in the dorsal portion of the spine. Therefore, a device inserted via posterior access will, in general, be smaller than the area of the vertebral body and will be asymmetrically positioned on the vertebral body. Therefore, in general, in order to provide the separation and cushioning capability of a healthy disc, at least two such devices may be implanted, each on a different side of the spinal cord.

Disc replacement devices and methods have been described previously. The described devices vary from one another with respect to how the devices are inserted into and maintained in their position between vertebral bodies. For example, devices described by Salib et al. (U.S. Pat. No. 5,258,031) and by David (WO 2006/049846) are attached by screws to vertebrae. Several devices, such as those described by Viart et al. (U.S. Pat. No. 6,682,562), Middleton (U.S. Pat. No. 6,136,031), Levieux (US 2008/0243253), and Albert et al. (US 2008/0065211), for example, are designed to be inserted via anterior access to the spine. Devices designed for posterior insertion, including tools to aid in the insertion, were described by Kuntz (U.S. Pat. No. 4,349,921) and Varga et al. (U.S. Pat. No. 6,852,127). Perren et al. (U.S. Pat. No. 6,019,793) describe a device made of shape memory alloy that is inserted into the spine in a compressed state, and regains its expanded state after insertion.

The various devices allow varying degrees of freedom of movement. For example, various components of a device may be free to rotate relative to one another about one or more axes, or to translate relative to one another along one or more axes. Such freedom of movement may facilitate implantation by enabling the device to accommodate itself to the shape of the surfaces surrounding it. In addition, such freedom of movement may enable a degree of movement of the vertebrae. For example, a healthy spine may enable bending in a ventral-dorsal direction (flexion-extension), lateral (right-left) bending, or rotation about its (superior-inferior) axis.

Various designs have been described which provide various degrees of freedom of movement. Some devices rely on elastic elements to provide a restoring force against compression. Elastic elements may also enable one or more degrees of freedom of rotation or translation. Such devices are described by, for example, by Viart et al. (annular element of visco-elastic material), Ratron (U.S. Pat. No. 5,676,702, elastically deformable connecting element), Rhoda et al. (US 2007/0010826, spring), Simonson (U.S. Pat. No. 6,572,653 and US 2004/181284, spring), Ralph et al. (US 2005/234554, slotted Belleville washer), and Bryan et al. (WO 00/13620, hemi-lunar resilient body).

Other devices rely of various forms of joints or joining elements to provide freedom of movement. Rhoda et al. and Buettner-Janz (US 2006/0235531), for example, describe devices containing slidable elements for providing translational or rotational motion. Other devices include domes or other projections with curved surfaces that are confined to matching sockets or grooves. Such devices are described by, for example, Eisermann et al. (WO 03/090648), Sears et al. (WO 2005/89680), Beaurain et al. (US 2004/0243240), Salib et al. David describes a ball bearing confined to a longitudinally oriented channel on one plate, and a transversely oriented channel on the other. Levieux describes a kidney-shaped recess to contain a curved cap. Errico et al. (WO 2004/028415) describe a projection with curved surfaces that is held in a socket by a housing. Van Hoeck et al. (WO 2006/107766) describe a device with plates provided with a dome and socket at their interface, where relative motion is limited by a tether. De Villiers et al. (WO 2006/014830) describe a device with two outer plates that are free to rotate about a curved central core element, with latching to limit the motion and to prevent separation of the plates.

It is an object of the present invention to provide a disc prosthetic implant device and method for posterior insertion, providing the vertebrae above and below the device with a suitable degree of freedom of motion

Other aims and advantages of the present invention will become apparent after reading the present invention and reviewing the accompanying drawings.

### SUMMARY OF THE INVENTION

There is thus provided, in accordance with some embodiments of the present invention, an intervertebral prosthetic implant device. The device includes a first plate with an axis support for supporting a rolling element substantially separate from the remainder of the first plate, designed to allow rolling of the rolling element. The device also includes a second plate that includes a groove designed to accommodate the rolling element, facilitating rotating and rolling motion of the second plate with respect to the first plate by rolling over the rolling element.

Furthermore, in accordance with some embodiments of the present invention, the device is designed to allow relative rotation between the first plate and the second plate so as to facilitate adjustment of outer surfaces of the device to corresponding vertebral end plates.

Furthermore, in accordance with some embodiments of the present invention, the groove is elongated, allowing translational relative motion between the plates.

Furthermore, in accordance with some embodiments of the present invention, the groove is laterally curved.

Furthermore, in accordance with some embodiments of the present invention, the device includes an engagement arrangement adapted to engage the plates so as to prevent the plates from separating and to enable relative freedom of motion between the plates.

Furthermore, in accordance with some embodiments of the present invention, the engagement arrangement comprises two opposite protrusions and matching opposite indentations, each protrusion located on one of the plates and having a widened end that is adapted to be loosely held by the matching indentation, which is located on the other plate.

Furthermore, in accordance with some embodiments of the present invention, the protrusions are located on one plate and the indentations are located on the other plate.

Furthermore, in accordance with some embodiments of the present invention, each of the outer surfaces includes a rough surface.

Furthermore, in accordance with some embodiments of the present invention, the rough surface includes projections.

Furthermore, in accordance with some embodiments of the present invention, the outer surfaces are each substantially rectangular.

Furthermore, in accordance with some embodiments of the present invention, the outer surfaces are each laterally curved.

There is further provided, in accordance with some embodiments of the present invention, a prosthetic implant device that includes a cylindrical barrel, two substantially opposite plates on either side of the cylindrical barrel, and adapted to rotate about the barrel, and at least one resilient element connecting the plates and the cylindrical barrel.

Furthermore, in accordance with some embodiments of the present invention, the barrel, the plates, and the resilient element are fashioned out of a single piece.

Furthermore, in accordance with some embodiments of the present invention, each of the outer surfaces includes a rough surface.

Furthermore, in accordance with some embodiments of the present invention, the rough surface includes projections.

Furthermore, in accordance with some embodiments of the present invention, the outer surfaces are each substantially rectangular.

Furthermore, in accordance with some embodiments of the present invention, the outer surfaces are each laterally curved.

### BRIEF DESCRIPTION OF THE DRAWINGS

In order to better understand the present invention, and appreciate its practical applications, the following Figures are provided and referenced hereafter. It should be noted that the Figures are given as examples only and in no way limit the scope of the invention. Like components are denoted by like reference numerals.

FIG. 1A illustrates two disc prosthetic implant devices in accordance with some embodiments of the present invention as placed on a vertebra.

FIG. 1B illustrates laterally curved disc prosthetic implant devices, in accordance with some embodiments of the present invention, as placed on a vertebral body.

FIG. 2 shows a disc prosthetic implant device with a ball-in-groove mechanism in accordance with an embodiment of the present invention.

FIG. 3A shows the disc prosthetic implant device of FIG. 2 with the groove plate removed.

FIG. 3B shows another view of the disc prosthetic implant device of FIG. 2, with the base plate removed.

FIG. 3C is a cutaway view through the ball axis of the disc prosthetic implant device of FIG. 2.

FIG. 4A shows a disc prosthetic implant device with an elastic member in accordance with an embodiment of the present invention.

FIG. 4B shows another view of the disc prosthetic implant device shown in FIG. 4A.

FIG. 5 illustrates a variant of the disc prosthetic implant device shown in FIG. 4A with two elastic members.

FIG. 6 shows two vertebrae with pedicle screws inserted.

FIG. 7 shows a distractor tool for manipulating vertebrae as attached to vertebrae.

FIG. 8 is a cutaway view of the vertebral distractor tool shown in FIG. 7.

FIG. 9A shows a prosthetic implant device insertion tool holding a disc prosthetic implant device in accordance with embodiments of the present invention.

FIG. 9B is a side view of the prosthetic implant device insertion tool.

### DETAILED DESCRIPTION OF EMBODIMENTS

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

A spinal disc prosthetic implant device, in accordance with embodiments of the present invention, is designed to replace an impaired intervertebral disc in a patient. In order to replace an impaired disc, the disc prosthetic implant device is implanted in the intervertebral gap between the vertebral bodies of two adjacent vertebrae. In general, two such devices are implanted in the intervertebral gap to enable symmetric cushioning and support of the adjacent vertebrae. Under some circumstances, for example, when implanted between vertebrae of the cervical spine, a single implanted prosthetic implant device may be sufficient. The spinal disc prosthetic implant device is designed to provide at least some of the function of a healthy intervertebral disc. In particular, the spinal disc prosthetic implant device is designed to provide the vertebrae on either side of the device with at least one degree of freedom of limited relative movement.

In general, a prosthetic implant device in accordance with embodiments of the present invention includes an upper plate or section, and a lower plate or section. When the prosthetic implant device is implanted between two vertebrae, the outer surfaces of the plates (the top surface of the upper plate and the bottom surface of the lower plate) are in contact with the vertebrae. The top surface of the upper plate is in contact with the bottom of the vertebral body that is above the prosthetic implant device. The bottom surface of the lower section is in contact with the top of the vertebral body that is below the prosthetic implant device. The outer surfaces of the plates may be rough or jagged. For example, the outer surfaces may be provided with projections. The purpose of the projections is to penetrate a small distance into the bony surface of a vertebral body. Such penetration into the bony surface of the vertebral bodies may assist the outer surfaces of the plates to adhere or bond to the vertebral bodies.

The upper and lower plates engage one another by an engagement arrangement or mechanism that enables a limited amount of relative translational or rotational motion between the plates. The limited relative motion may enable the outer surfaces of the plates to conform to, and maintain maximum contact with, the bony surfaces of the vertebral bodies adjacent to the implanted prosthetic implant device. Adapting the shape of the device to the bony surfaces of the vertebral bodies may enable good contact between the device and the vertebral bodies. In addition, the limited relative motion between the plates may provide the vertebrae above and below the prosthetic implant device with at least some of the freedom of movement enabled by a healthy disc.

Two or more disc prosthetic implant devices may be implanted between a single pair of vertebral bodies, situated laterally to one another. When two or more devices are implanted, relative motion between the vertebrae may be limited by the combined limits of the motion of the implanted devices. For example, although a single device may rotate about a given axis, the combined limits of motion of the implanted devices may prevent a similar relative motion of the adjacent vertebrae.

The prosthetic implant device may be constructed of suitable biocompatible, durable, and sterilizable materials. An example of such a material is BioDur® CCM® alloy. The outer and other surfaces of the device may be coated with a suitable material. For example, surfaces may be coated with porous titanium or hydroxylapatite in order to promote bone growth.

Disc prosthetic implant devices in accordance with embodiments of the present invention may be inserted between the vertebral bodies of two adjacent vertebrae of a patient. A procedure for insertion of the devices may be selected to minimize the invasiveness of the procedure. For example, the disc prosthetic implant devices may be inserted from the dorsal side of the patient, posterior to the vertebrae (posterior access). Two prosthetic implant devices may be inserted, one prosthetic implant device on each side (left and right) of the vertebral body. The insertion path may be selected lateral to the vertebral foramen, avoiding traversing the vertebral foramen and risking damage to the spinal cord. Another possibility is to insert the prosthetic implant devices via the anterior side of the vertebral bodies (anterior access). Anterior access may require traversing the body of the patient from anterior to posterior, and may be, therefore, more invasive than posterior access.

A procedure for inserting disc prosthetic implant devices in via posterior access, in accordance with embodiments of the present invention, may proceed as follows: The back of the patient in the vicinity of an impaired intervertebral disc is opened. Tissue covering the vertebrae adjacent to the disc may be displaced in order to enable direct access to the intervertebral space. A pedicle screw is inserted into each pedicle, right and left, of the vertebra superior to and of the vertebra inferior to the intervertebral disc being replaced. A pedicle screw adapter is attached to the distal end of each pedicle screw. A vertebrae distractor tool is mounted on the pair of right pedicle screw adapters. A similar vertebrae distractor tool is mounted on the pair of left pedicle screw adapters. Alternatively, a single tool including two connected distractor tools may be mounted on the four pedicle screws. The distractor tool may then be adjusted so as to manipulate the attached vertebral bodies. The distractor tools may be adjusted so as rotate the ends of the vertebral bodies so that they are approximately parallel, and to increase the intervertebral gap between the two vertebrae. Such manipulation of the vertebral bodies may facilitate insertion of prosthetic implant devices into the intervertebral gap, and may prevent injury to the surfaces of the vertebral bodies from the insertion procedure.

A partial laminectomy may be performed to remove one or more sections of the lamina of the vertebrae may be removed. The sections of the lamina are removed in order to create an opening for insertion of each prosthetic implant device. Alternatively, an insertion path lateral to the lamina and the pedicles may be selected. With such a lateral insertion path, performance of a partial laminectomy may not be necessary.

A portion of the tissue of the impaired disc may also be removed to make room for the prosthetic implant device. Each disc prosthetic implant device is inserted between the vertebral bodies using an insertion tool. Placement of the prosthetic implant device may be guided by concurrently acquired x-ray, or x-ray fluoroscopy, images. When each prosthetic implant device is properly situated between the intervertebral bodies, the insertion handle is manipulated to release the prosthetic implant device. When all inserted prosthetic implant devices are properly situated, the distractor tool is adjusted so as to release the vertebrae and allow the space between the vertebral bodies to close. Releasing the vertebrae may cause the vertebrae to hold the prosthetic implant devices firmly in place. Tissue displaced from the vertebrae may be allowed to return to its place, and the opening in the back of the patient is closed.

Reference is now made to the figures.

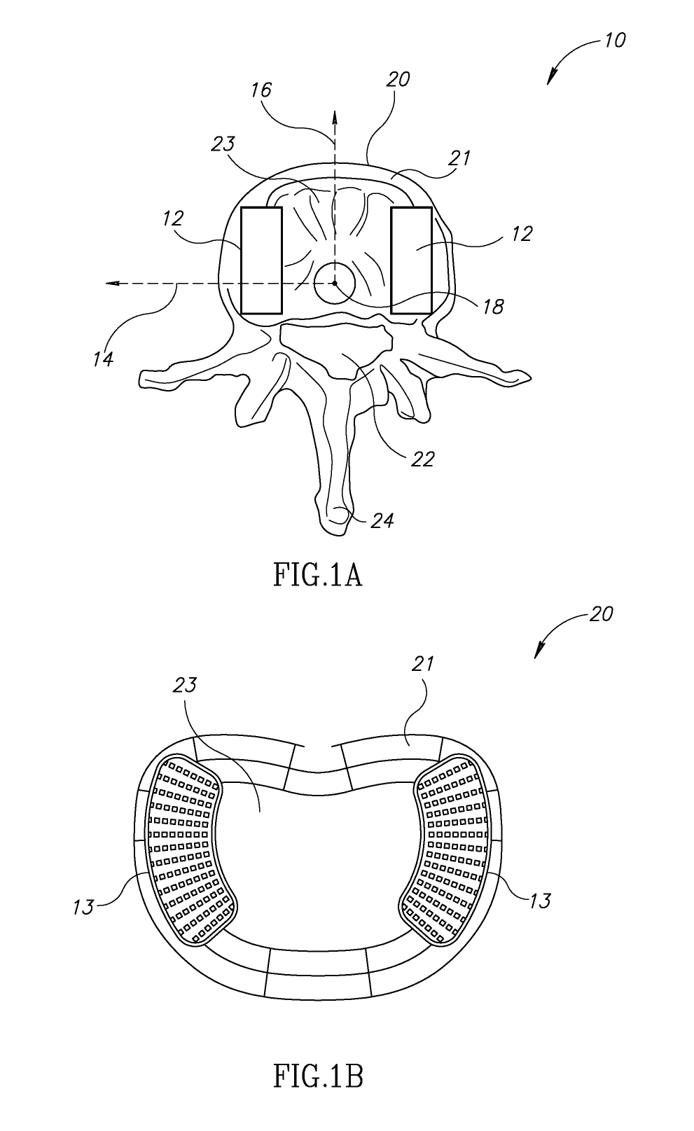

FIG. 1A illustrates two disc prosthetic implant devices in accordance with some embodiments of the present invention as placed on a vertebra. Vertebra 10 is viewed from above. Vertebral body 20 is ventral to spinous process 24. As shown in FIG. 1A, two disc prosthetic implant devices 12 are positioned on vertebral body 20. When disc prosthetic implant devices 12 are implanted between two vertebral bodies, a second vertebral body (not shown) will be positioned above disc prosthetic implant devices 12 and vertebral body 20. An insertion path for inserting disc prosthetic implant devices 12 avoids traversing vertebral foramen 22. The spinal cord (not shown) passes through vertebral foramen 22 in a direction approximately perpendicular to the plane of the Figure.

The perimeter of disc prosthetic implant device 12 may be approximately rectangular when viewed from above as in FIG. 1A. Alternatively, part of the perimeter may be laterally curved, for example, with a concave side and a convex side. FIG. 1B illustrates laterally curved disc prosthetic implant devices, in accordance with some embodiments of the present invention, as placed on a vertebral body. A curved concave-convex perimeter may enable placement of a large part of curved prosthetic implant device 13 on cortical rim 21 of vertebral body 20. Cortical rim 21 is harder than cancellous bone 23 of vertebral body 20 that is located interior to cortical rim 21. Therefore, confining placement of curved prosthetic implant device 13 to cortical rim 21 may prevent possible subsidence of curved prosthetic implant device 13 into cancellous bone 23. Preventing subsidence may increase the ability of curved prosthetic implant device 13 to separate the vertebral bodies for an extended period of time.

Three axes are defined in FIG. 1A: lateral axis 14, ventral-dorsal axis 16, and cranial-caudal axis 18 (perpendicular to the plane of FIG. 1A). Various embodiments of a disc prosthetic implant device may enable relative rotational and translational motion between upper and lower sections of the device. The amount of enabled rotational motion about the various axes, and translational motion along the various axes, may vary among embodiments of the present invention. In addition, when two devices are implanted between two vertebrae, constraints on the motion of the combination of the two devices may impose further constraints on the relative motion of the vertebrae.

In particular, a prosthetic implant device in accordance with embodiments of the present invention may enable some rotation in the sagittal plane about lateral axis 14, corresponding to flexion-extension of the spine. However, the device may enable little or no lateral bending in the coronal plane about ventral-dorsal axis 16, or translation along lateral axis 14. A prosthetic implant device in accordance with some embodiments of the present invention may enable limited relative translation between its upper and lower sections along ventral-dorsal axis 16. When two or more such devices are implanted between two vertebrae and aligned such that their translation axes are oriented tangentially, a limited amount of relative rotation of the vertebrae in the transverse plane about cranial-caudal axis 18 may be enabled. A prosthetic implant device in accordance with other embodiments of the present invention, however, may enable little or no relative translational motion between its upper and lower sections along ventral-dorsal axis 16. Implantation of two such prosthetic implant devices may then prevent rotation of the adjacent vertebrae in the transverse plane about cranial-caudal axis 18.

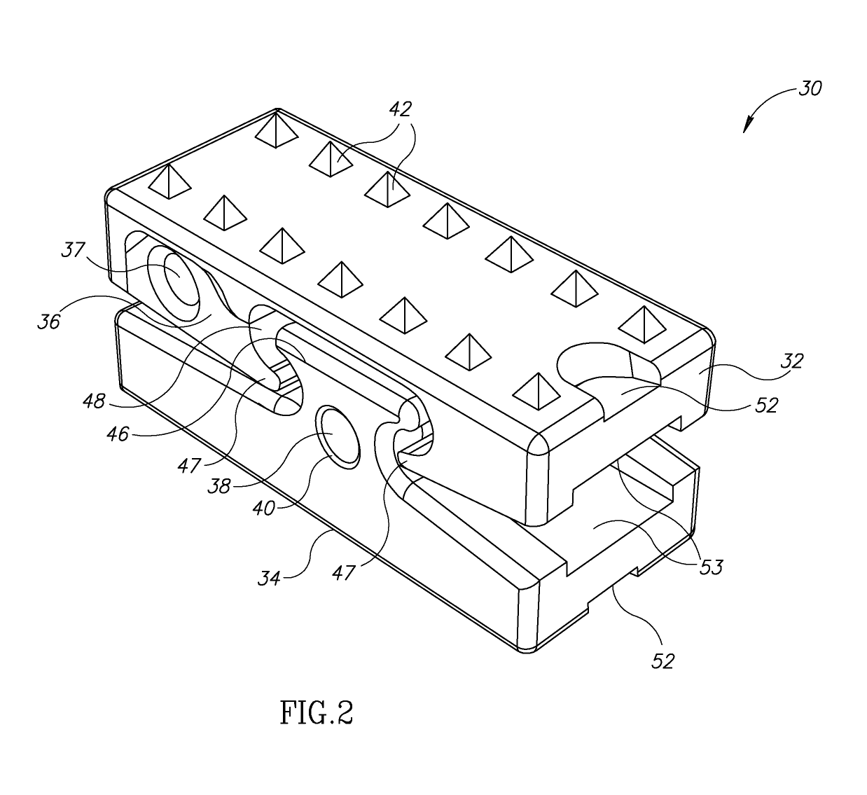

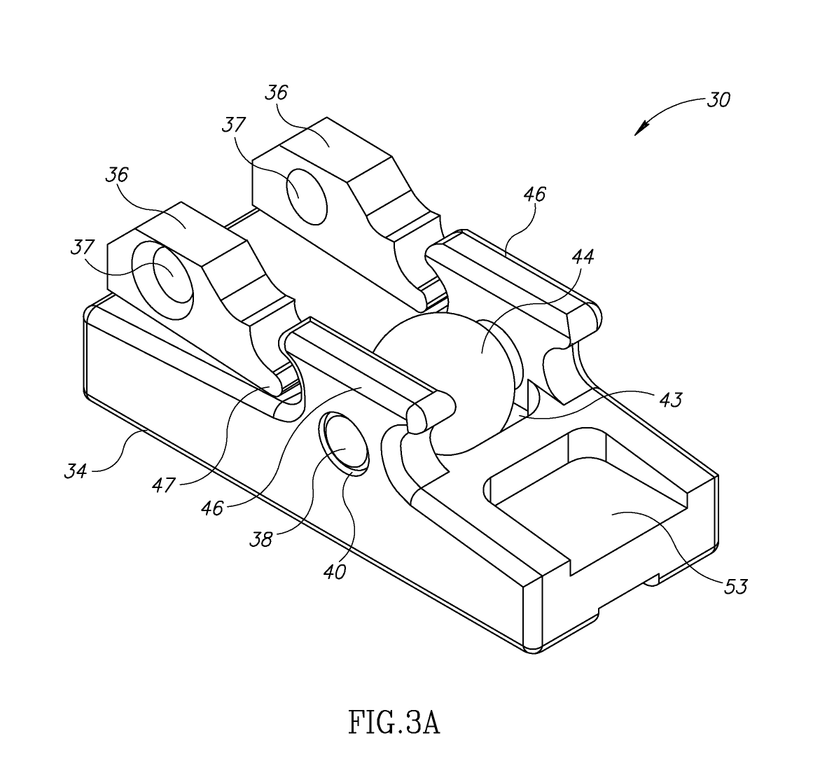

FIG. 2 shows a disc prosthetic implant device with a ball-in-groove mechanism in accordance with an embodiment of the present invention. Ball-in-groove prosthetic implant device 30 includes a groove plate 32 and a base plate 34. The outer surfaces of groove plate 32 and base plate 34 (the outer surface of base plate 34 is not visible in FIG. 2) are partially covered with projections 42. Each outer surface is designed to contact a bony surface of a vertebral body when ball-in-groove prosthetic implant device 30 is inserted between two vertebral bodies. Projections 42 may assist in enabling each outer surface to adhere or bond to the bony surface of a vertebral body. Such projections may have features to enhance their ability to allow ingrowth of bone tissue or otherwise become better fixated in the bony surface of the vertebral body. Such features may include coatings or surface structure. For example, outer surfaces and projections that are intended for contact with a bony surface of a vertebral body may be coated with porous titanium or hydroxylapatite.

A protrusion or extension with a widened end, such as tongue 46, is located on one plate. The widened end of tongue 46 is designed to fit loosely into a recess or indentation on the other plate, such as partially closed groove 48. Partially closed groove 48 is formed by extensions 47 of groove plate 32 and locking panel 36. Two such protrusions may be located on opposite sides of one plate, and indentations designed to accommodate the indentations may by located on opposite sides of the other plate. Alternatively, on each plate, a protrusion may be located on one side, and an indention on the other side.

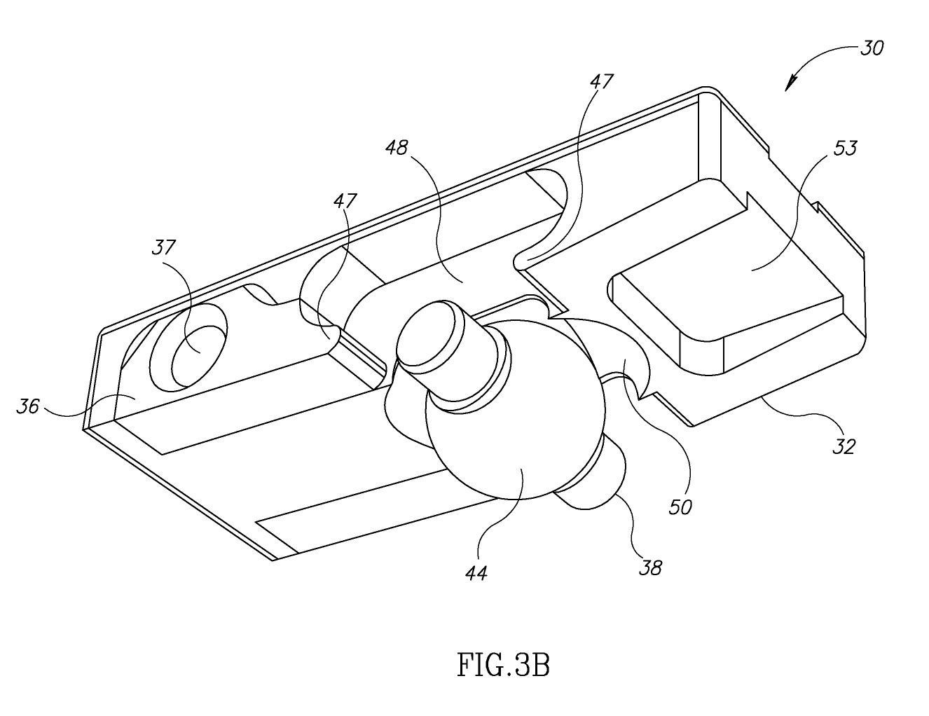

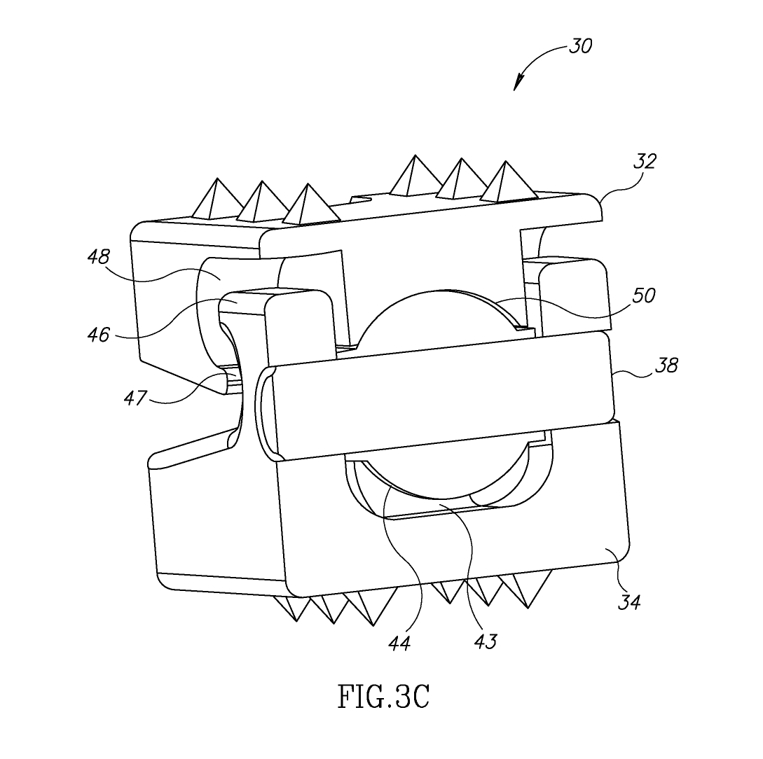

The loose fit between tongue 46 and partially closed groove 48 enables a limited amount of relative translational and rotational motion between groove plate 32 and base plate 34. Relative motion is also enabled and limited by a ball-in-groove mechanism (seen in FIG. 3A and FIG. 3B and described below). During construction of ball-in-groove prosthetic implant device 30, locking panel 36 is attached to groove plate 32 after tongue 46 has been inserted into the location of groove 48. Attachment of locking panel 36 to groove plate 32 then retains tongue 46 inside partially closed groove 48, preventing separation of groove plate 32 from base plate 34. Locking panels 36 may be bolted to groove plate 32 by means of a suitable mechanism such as, for example, a bolt, screw, or rivet inserted through locking panel opening 37.

FIG. 3A shows the disc prosthetic implant device of FIG. 2 with the groove plate removed. FIG. 3B shows another view of the disc prosthetic implant device of FIG. 2, with the base plate removed. (It should be noted that the removal of components in FIG. 2 and FIG. 3A is for illustrative purposes only, and may not represent an attainable physical configuration.) FIG. 3C is a cutaway view through the ball axis of the disc prosthetic implant device of FIG. 2. FIG. 2 and FIG. 3A illustrate the ball-in-groove mechanism. An axis of a rolling element, such as ball 44, is mounted on axle 38. Axle 38 passes through the center of ball 44. Alternatively, the rolling element may be barrel-shaped, cylindrical, or conical, or have any other shape with a substantially circular cross section. The relative freedom of motion of the device may depend of the degree of symmetry of the rolling element. For example, a ball-shaped rolling element may enable a greater number of degrees of freedom of motion than a cylindrical rolling element.

Axle 38 is held within openings 40 on tongues 46 of base plate 34, such that ball 44 is free to rotate about the axis defined by axle 38. In particular, axle 38 holds ball 44 such that the surface of ball 44 substantially separate from the remainder of base plate 34. That is, the surface of ball 44 does not contact any surface of ball recess 43, or any other internal surface of base plate 34.

Ball 44 fits within, and is in contact with an inner surface of, ball groove 50 on groove plate 32. Ball 44 is contained within ball groove 50. Ball groove 50 may be elongated and may be laterally curved, for example, with a convex side and a concave side. Thus, the assembly of ball 44 in ball groove 50 may enable a limited degree of relative rotational and translational motion between groove plate 32 and base plate 34. Relative motion between groove plate 32 and base plate 34 may be enabled by rolling motion of ball groove 50 over ball 44, or by rotation of ball 44 about axle 38. In particular, the ball-in-groove mechanism limits translational motion in the direction parallel to axle 38 (corresponding to lateral axis 14 in FIG. 1A, when implanted in a spine). However, smooth rocking motion of groove plate 32 relative to base plate 34 is enabled about an axis parallel to axle 38 (corresponding approximately to rotation about lateral axis 14, when implanted in a spine, corresponding to flexion-extension of the spine). For example, an embodiment of the device may enable a relative rotation between groove plate 32 and base plate 34 about axle 38 of about 6° to one side, and of about 12° to the other.

When ball-in-groove prosthetic implant device 30 is implanted in a spine between two vertebral bodies of two vertebrae, the limited relative rotational and translational motion enable limited relative motion between the two vertebrae. The limited relative motion may serve to restore at least partially the function of vertebrae separated by a healthy intervertebral disc. For example, enabled rotation about axle 38 may restore partially or completely the range of flexion-extension motion of the vertebrae. As another example, relative rotation between groove plate 32 and base plate 34 of each of the two devices may enable lateral rotation of the spine about cranial-caudal axis 18 in FIG. 1A. For example, some embodiments of the device may enable a relative lateral rotation of about ±3° to ±8° between vertebral bodies separated by two aligned devices.

As shown in FIG. 3A, FIG. 3B, and FIG. 3C, axle 38 is ranged with its axis perpendicular to the long dimension of ball-in-groove prosthetic implant device 30. The long axis of ball groove 50 is oriented longitudinally, parallel to the long dimension of ball-in-groove prosthetic implant device 30. The longitudinal orientation of ball groove 50 may enable a degree of relative translational motion along an axis parallel to the length of the groove. Implantation of two or more such devices between vertebral bodies, each with axle 38 oriented radially to a vertebral rotation axis and the translation axis oriented tangentially, may enable the spine to rotate in a radial plane (about cranial-caudal axis 18 in FIG. 1A). Alternatively, the axes of the axle and groove may be oriented obliquely to the sides of the prosthetic implant device. Such an oblique arrangement may be advantageous, for example, when a prosthetic implant is implanted such that its sides are oriented obliquely to the ventral-dorsal axis 16 (FIG. 1A).

The enabled rotational and translational motion of the plates of ball-in-groove prosthetic implant device 30 may aid the implantation of ball-in-groove prosthetic implant device 30 between vertebral bodies. During implantation, the limited motion of groove plate 32 and base plate 34 may enable the outer surface of groove plate 32 or base plate 34 to rotate so as to accommodate local variations in a bony surface of an adjacent vertebral body. Where such local variations occur, after implantation of ball-in-groove prosthetic implant device 30 each outer surface of the device may be substantially parallel to an adjacent section of the bony surface of the vertebral body. However, the outer surfaces of an implanted ball-in-groove prosthetic implant device 30 may not be parallel to each other.

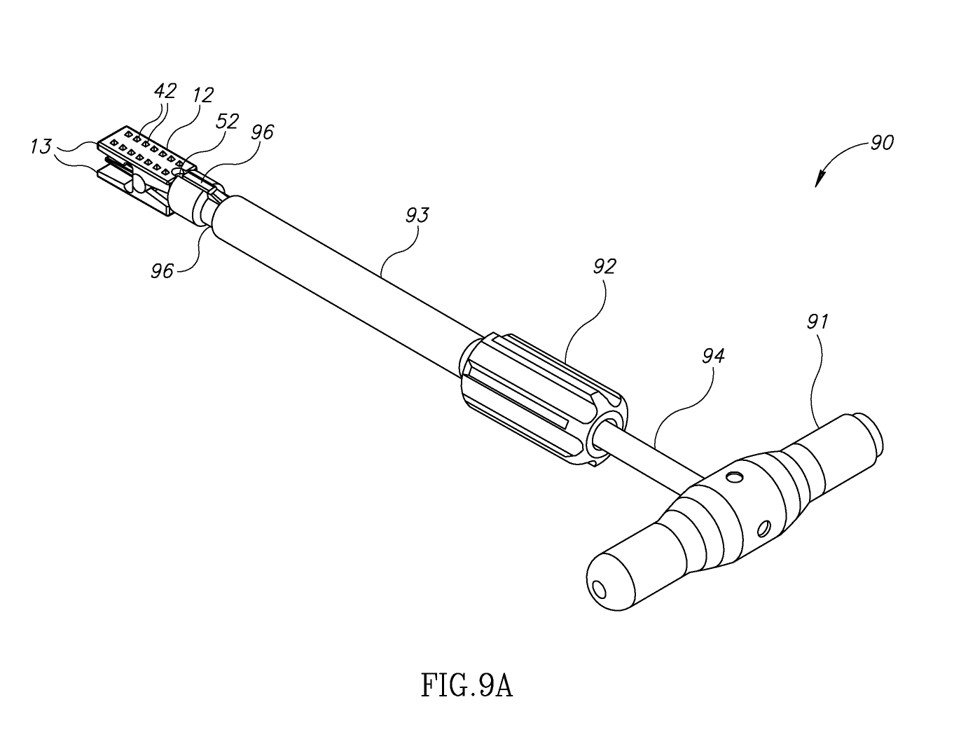

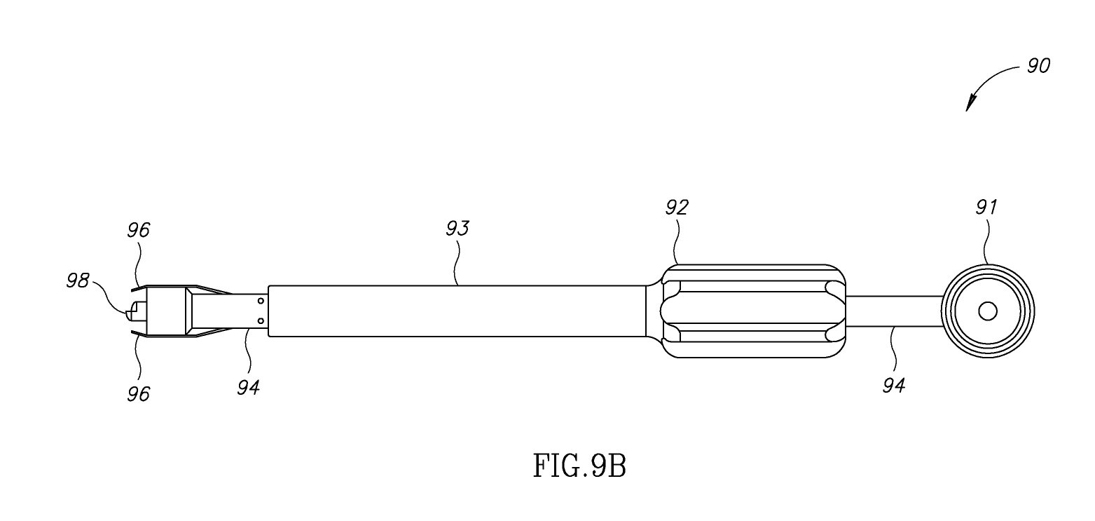

Groove plate 32 and base plate 34 are each provided with an outer recess 52 and an inner recess 53. Outer recesses 52 are shaped so as to match fingers 96 (shown in FIG. 9B) of prosthetic implant device insertion tool 90 (FIG. 9B). Inner recesses 53 are shaped to fit over central projection 98 (FIG. 9B) of prosthetic implant device insertion tool 90. Fingers 96 of prosthetic implant device insertion tool 90 may be manipulated to grip outer recesses 52. Fingers 96 hold inner recesses 53 of ball-in-groove prosthetic implant device 30 against central projection 98. When thus held, the outward facing surfaces of groove plate 32 and base plate 34 are substantially parallel to one another. Once ball-in-groove prosthetic implant device 30 is inserted and properly positioned between vertebral bodies, fingers 96 of the prosthetic implant device insertion tool may be manipulated to release outer recesses 52.

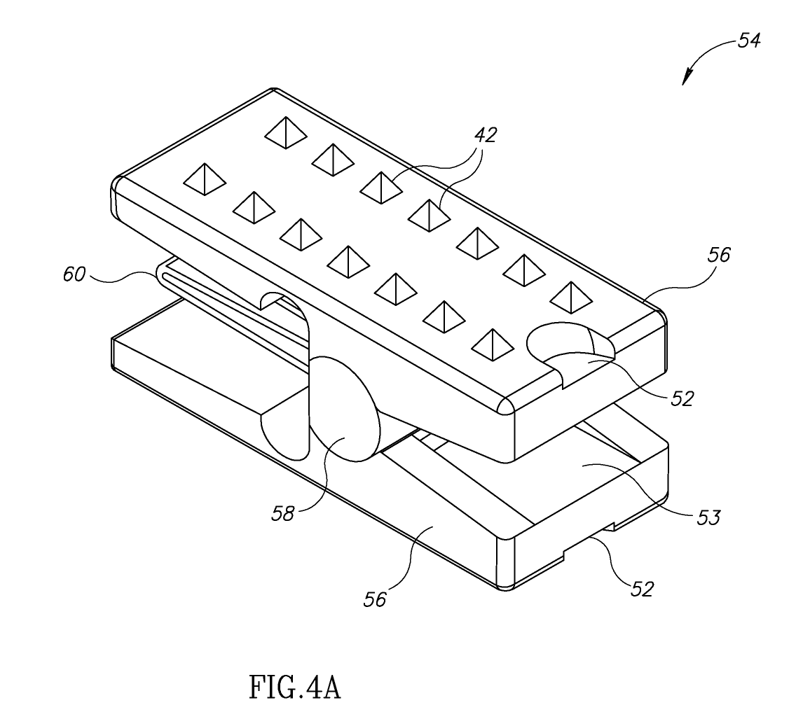

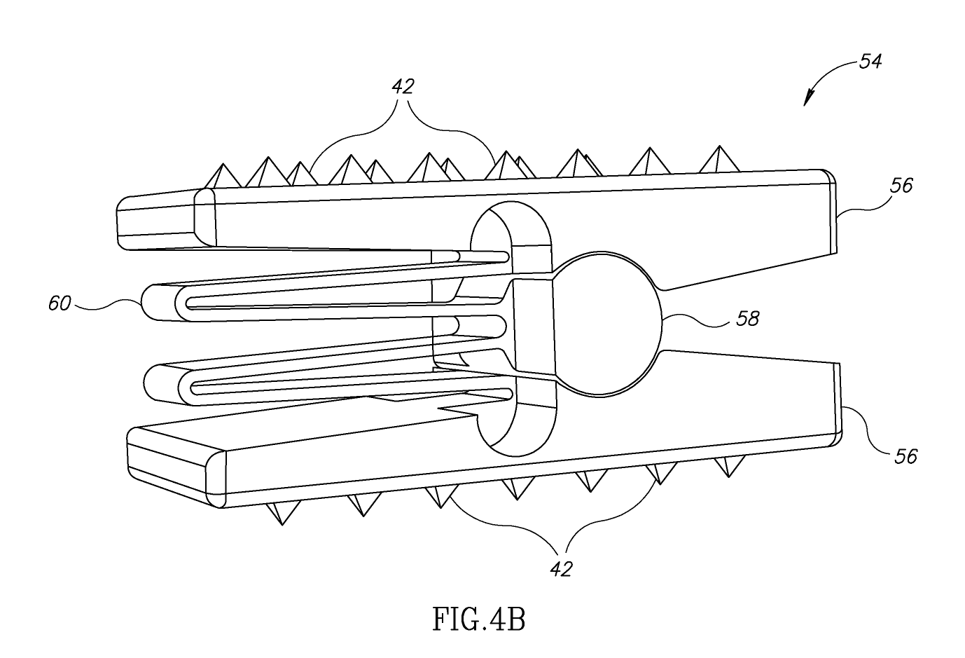

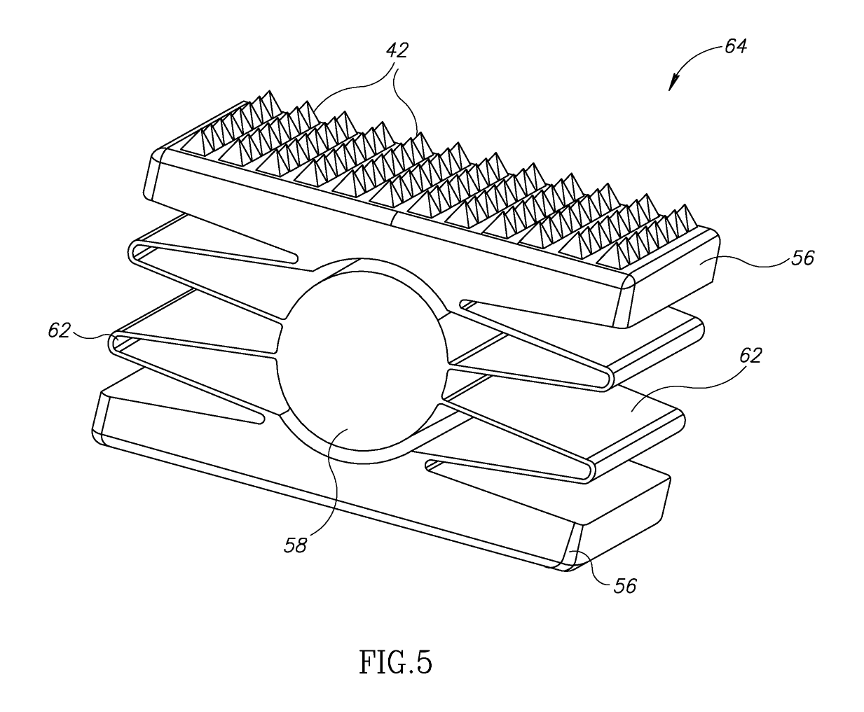

In other embodiments of the present invention, the mechanism for holding the plates together may vary, as well as the mechanism for enabling relative motion between the plates. FIG. 4A shows a disc prosthetic implant device with an elastic member in accordance with an embodiment of the present invention. FIG. 4B shows another view of the disc prosthetic implant device shown in FIG. 4A. Elastic member prosthetic implant device 54 includes two plates 56. The outer surfaces of plates 56 are partially covered with projections 42. Each outer surface is designed to contact a bony surface of a vertebral body when elastic member prosthetic implant device 54 is inserted between two vertebral bodies. Applying an appropriate torque to plates 56 may cause plates 56 to rotate relative to one another about the axis of cylindrical axis rod 58 (corresponding approximately to lateral axis 14 in FIG. 1A, when implanted in a spine, corresponding to flexion-extension of the spine). The extent of this relative rotational motion is limited by the shape of plates 56. Only this one rotational degree of freedom is enabled by elastic member prosthetic implant device 54. A resilient elastic member 60 is connected to plates 56 and to a cylindrical barrel or axis rod 58. In general, axis rod 58 may be substantially cylindrical. For example, the cross section of axis rod 58 may be generally circular but missing a segment. Elastic member 60 holds plates 56 and cylindrical axis rod 58 together to form a single unit, elastic member prosthetic implant device 54. Plates 56, elastic member 60, and cylindrical axis rod 58 may, for example, be fashioned, shaped, or otherwise manufactured from a single piece of material. For example, the fashioning process may include wire-cutting the piece of material to form the various components of elastic member prosthetic implant device 54. When rotation about the axis of cylindrical axis rod 58 takes place, elastic member 60 may apply an elastic restoring torque to plates 56. The elastic restoring torque tends to restore plates 56 to their unloaded positions when the torque that caused the relative motion is reduced.

FIG. 5 illustrates a variant of the disc prosthetic implant device shown in FIG. 4A with two elastic members. Dual elastic member prosthetic implant device 64 includes two elastic members 62 arranged symmetrically about cylindrical axis rod 58. Such an arrangement may prevent separation of plates 56 from one another, or to increase, or ensure symmetric application of, the elastic restoring torque.

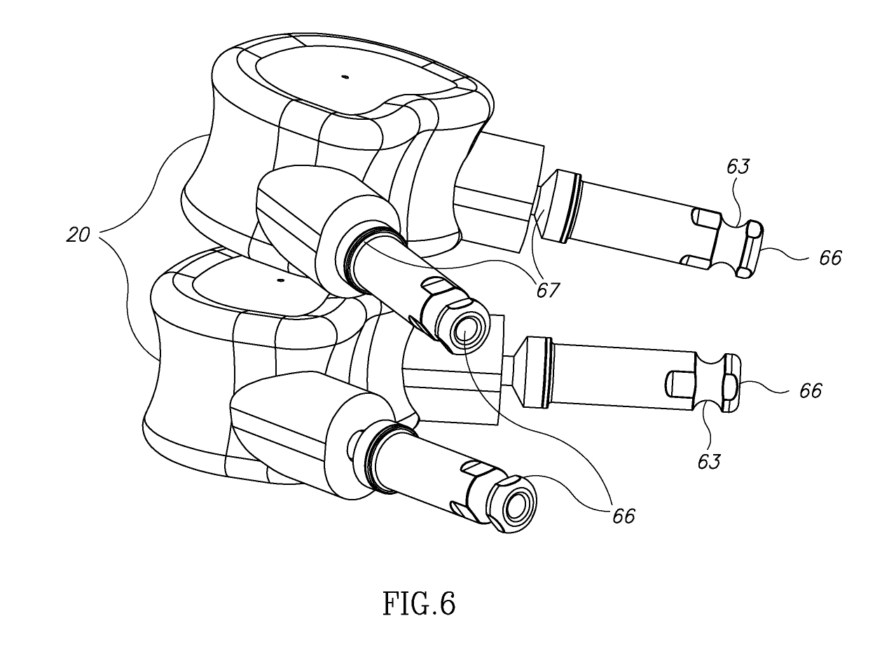

A procedure for implantation of a disc prosthetic implant device in accordance with embodiments of the present invention is now described. The disc prosthetic implant device may be inserted between the vertebral bodies of two vertebrae between which the function of intervertebral disc is impaired. Posterior insertion of the disc prosthetic implant device may be considered to be less invasive than anterior insertion. Posterior insertion of a disc prosthetic implant device may proceed as follows: At least a posterior section of each of the two vertebrae may be exposed by making a dorsal incision and displacing tissue covering the posterior side of the vertebrae. Pedicle screws are inserted, using standard pedicle screw insertion techniques, into the vertebral bodies of the two vertebrae. In general, two screws may be inserted into each vertebral body, one via each pedicle of the vertebrae. FIG. 6 shows two vertebrae with pedicle screws inserted. Pedicle screws 67 are inserted into vertebral bodies 20. Pedicle screw adapters 66 are screwed on to the distal ends of pedicle screws 67.

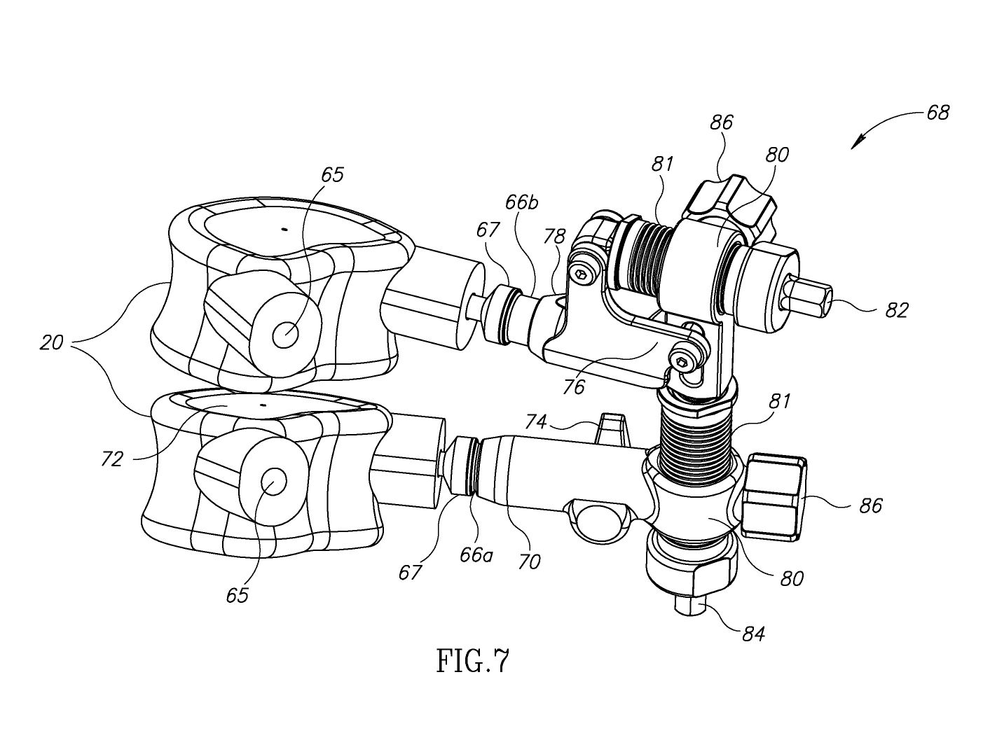

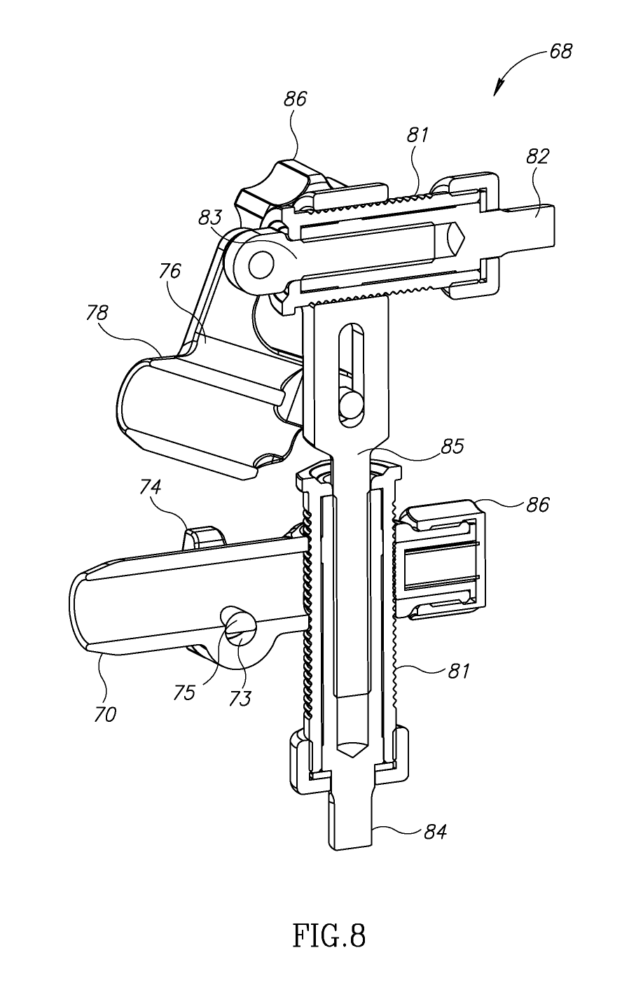

FIG. 7 shows a distractor tool for manipulating vertebrae as attached to vertebrae. FIG. 8 is a cutaway view of the vertebral distractor tool shown in FIG. 7. Distractor tool 68 is designed to attach to pedicle screw adapters 66a and 66b. Handles 86 may be loosened to allow sleeves 80 to slide freely over grooved tube surfaces 81. Free sliding of sleeves 80 enables free manipulation of distractor tool 68 for attachment to pedicle screw adapters 66a and 66b. Once distractor tool 68 is attached to pedicle screw adapters 66a and 66b, distractor tool 68 may adjusted so as to manipulate pedicle screws 67. Manipulating pedicle screws 67 manipulates vertebral bodies 20 into which pedicle screws 67 are inserted. Manipulation of vertebral bodies 20 may include reorienting the facing surfaces vertebral bodies 20 so as to be substantially parallel to one another. Manipulation of vertebral bodies 20 may also include expanding intervertebral gap 72 by an amount sufficient to enable insertion of a disc prosthetic implant device into the gap. A substantially parallel orientation of the surfaces of vertebral bodies 20 may facilitate insertion of the disc prosthetic implant device. In general, a second distractor tool (not shown in order to simplify the Figure) may be concurrently attached to a second pair of pedicle screws (not shown) inserted into holes 65. The second distractor tool may be adjusted concurrently with distractor tool 68. Alternatively, the distractor tool may include two connected sections, each section corresponding functionally to a separate distractor tool 68. Connecting the section may ensure that as a result of manipulation of the right and left sets of pedicle screws, the vertebral bodies maintain a desired relative spatial placement.

Screw gripping tube 70 fits over pedicle screw adapter 66a. In general, the orientation of pedicle screw adapter 66b will not be parallel to the orientation of pedicle screw adapter 66a. However, by manipulating sleeves 80, tube 78 may be fit concurrently over pedicle screw adapter 66b. Screw gripping tube 70 is locked onto pedicle screw adapter 66a by means of tube locking handle 74. By turning tube locking handle 74, projection 75 is rotated out of indentation 73 so as to fit into recessed groove 63 (shown in FIG. 6) of pedicle screw adapter 66a.

Once tubes 70 and 78 are properly attached to pedicle screw adapters 66a and 66b, handles 86 may be tightened to prevent free movement of components of distractor tool 68. Stems 82 and 84 may be turned to manipulate pedicle screw adapters 66a and 66b. Turning stem 82 causes threaded rod 83 to extend or retract, manipulating the orientation of tube 78. Turning stem 84 causes threaded rod 85 to extend or retract, changing the separation distance between tube 70 and tube 78. Pedicle screws inserted into holes 65 may be manipulated by similar components of a second distractor tool (not shown). Manipulating pedicle screw adapters 66a and 66b manipulates the positions of vertebral bodies 20, adjusting the size and shape of intervertebral gap 72. For example, stem 82 may be turned in order to rotate vertebral bodies 20 so as to make the top and bottom surfaces of intervertebral gap 72 parallel to one another. Stem 84 may then be turned in order to increase the height of intervertebral gap 72. A partial laminectomy may be performed to remove a portion of the lamina of each of the vertebrae adjacent to intervertebral gap 72. In addition, at least some of disc tissue filling intervertebral gap 72, may be removed to enable insertion of a prosthetic implant device.

A prosthetic implant device insertion tool may then be used to insert a disc prosthetic implant device into the intervertebral gap. FIG. 9A shows a prosthetic implant device insertion tool holding a disc prosthetic implant device in accordance with embodiments of the present invention. FIG. 9B is a side view of the prosthetic implant device insertion tool. Prosthetic implant device insertion tool 90 includes a handle 91 for holding and manipulating prosthetic implant device insertion tool 90. Fingers 96 are designed to fit into outer recesses 52 of disc prosthetic implant device 12. (Disc prosthetic implant device 12 may represent any disc prosthetic implant device in accordance with embodiments of the present invention. FIG. 9A shows an elastic member prosthetic implant device as an example only.) Central projection 98 is inserted between plates 13 of disc prosthetic implant device 12. Sleeve 93 may be moved back and forth along tube 94. For example, sleeve 93 may be tapped and tube 94 may be threaded so that rotating handle 92 moves sleeve 93 back and forth along tube 94. When the end of sleeve 93 presses against fingers 96, fingers 96 are held fast against outer recesses 52, with central projection 98 applying a counterforce. In this manner, prosthetic implant device insertion tool 90 firmly grips disc prosthetic implant device 12. When so gripped, the outward facing surfaces of disc prosthetic implant device 12 may be substantially parallel to one another. Prosthetic implant device insertion tool 90 may then manipulate disc prosthetic implant device 12 to a desired location within the intervertebral gap between two vertebral bodies.

When disc prosthetic implant device 12 has been inserted into a desired location, sleeve 93 may be retracted from fingers 96 by rotating handle 92. When sleeve 93 is retracted from fingers 96, fingers 96 no longer apply force to outer recesses 52. At this point, pulling backward on handle 91 may remove prosthetic implant device insertion tool 90 from disc prosthetic implant device 12, leaving disc prosthetic implant device 12 in place. Distractor tool 68 (FIG. 7) may then be adjusted so as to enable the intervertebral gap to close. The surfaces of the vertebral bodies on either side of the intervertebral gap may then press against the outward facing surfaces on disc prosthetic implant device 12. Projections 42 on the outward facing surfaces may penetrate the bony surfaces of the vertebral bodies, holding disc prosthetic implant device 12 in place. Distractor tool 68 may then be removed from pedicle screw adapters 66 (FIG. 6), and pedicle screw adapters 66 may be removed from pedicle screws 67. Pedicle screws 67 may be removed form vertebral bodies 20, or left in place for possible future use.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the present invention.

1. An intervertebral prosthetic implant device comprising:
a first plate with an axis support for supporting a rolling element substantially separate from the remainder of the first plate, designed to allow rolling of the rolling element; and
a second plate that includes a groove designed to accommodate the rolling element, facilitating rotating and rolling motion of the second plate with respect to the first plate by rolling over the rolling element.

2. A device as claimed in claim 1, designed to allow relative rotation between the first plate and the second plate so as to facilitate adjustment of outer surfaces of the device to corresponding vertebral end plates.

3. A device as claimed in claim 1, wherein the groove is elongated, allowing translational relative motion between the plates.

4. A device as claimed in claim 3, wherein the groove is laterally curved.

5. A device as claimed in claim 1, comprising an engagement arrangement adapted to engage the plates so as to prevent the plates from separating and to enable relative freedom of motion between the plates.

6. A device as claimed in claim 5, wherein the engagement arrangement comprises two opposite protrusions and matching opposite indentations, each protrusion located on one of the plates and having a widened end that is adapted to be loosely held by the matching indentation, which is located on the other plate.

7. A device as claimed in claim 6, wherein the protrusions are located on one plate and the indentations are located on the other plate.

8. A device as claimed in claim 1, wherein each of the outer surfaces comprises a rough surface.

9. A device as claimed in claim 8, wherein the rough surface comprises projections.

10. A device as claimed in claim 1, wherein the outer surfaces are each substantially rectangular.

11. A device as claimed in claim 1, wherein the outer surfaces are each laterally curved.

12. A prosthetic implant device comprising:
a cylindrical barrel;
two substantially opposite plates on either side of the cylindrical barrel, and adapted to rotate about the barrel; and
at least one resilient element connecting the plates and the cylindrical barrel.

13. A device as claimed in claim 12, wherein the barrel, the plates, and said at least one resilient element are fashioned out of a single piece.

14. A device as claimed in claim 12, wherein each of the outer surfaces comprises a rough surface.

15. A device as claimed in claim 14, wherein the rough surface comprises projections.

16. A device as claimed in claim 12, wherein the outer surfaces are each substantially rectangular.

17. A device as claimed in claim 12, wherein the outer surfaces are each laterally curved.
